(19) United States
(12) Patent Application Publication
(10) Pub. No.: US 2019/0071645 A1
(43) Pub. Date: Mar. 7, 2019

(54) EFFICIENT DIFFERENTIATION OF HUMAN STEM CELLS TO DEFINITIVE ENDODERM

(71) Applicant: Sergio Mora, San Diego, CA (US)

(72) Inventor: Sergio Mora, San Diego, CA (US)

(21) Appl. No.: 16/125,184

(22) Filed: Sep. 7, 2018

Related U.S. Application Data
(60) Provisional application No. 62/555,259, filed on Sep. 7, 2017.

(51) Int. Cl. C12N 5/071; C07K 14/62

(52) U.S. Cl. CPC C12N 5/0677; C12N 5/0678; C07K 14/62; C12N 5/0613; C12N 2501/16; C12N 2500/25; C12N 2500/32; C12N 2500/46; C12N 2510/02

(57) ABSTRACT

The present disclosure provides a medium for cells, such as human stem cells including human induced pluripotent stem cells (iPScs) which medium enhances the formation of definitive endoderm (DE) which in turn can be subsequently directed and differentiated into mature cell types, e.g., pancreas insulin producing cells.

### CROSS-REFERENCE TO RELATED APPLICATIONS

This application claims the benefit of the filing date of U.S. application Ser. No. 62/555,259, filed on Sep. 7, 2017, the disclosure of which is incorporated by reference herein.

### BACKGROUND

The potential of human induced pluripotent stem (iPS) cells to differentiate into cell types of a variety of organs has generated much excitement over the possible use of iPS cells in therapeutic applications. Of great interest are organs derived from definitive endoderm, such as the pancreas.

The definitive endoderm gives rise to the epithelial lining of the respiratory and digestive tracts and to the thyroid, thymus, lungs, liver, and pancreas. It is formed during gastrulation, in which pluripotent epiblast cells are allocated to the three principal germ layers, ectoderm, mesoderm and definitive endoderm. The initiation of this process is evidenced by the appearance of the primitive streak in the posterior epiblast. As epiblast cells ingress through the primitive streak they undergo an epithelial-to-mesenchymal transition (EMT) and become either mesoderm or definitive endoderm. Although it has been suggested that these two cell types arise from a common precursor in the primitive streak, called the mesendoderm, the existence of a single embryonic cell with bipotent properties has not been proven in mammals.

### SUMMARY

The present disclosure focuses on directing stem cells, e.g., human stem cells including human iPS cells, to the definitive endoderm lineage prior to efficient differentiation to mature endoderm derivatives. The process of definitive endoderm formation in differentiating stem cell cultures, e.g., iPS cell cultures, includes an apparent epithelial-to-mesenchymal transition and a dynamic gene expression profile that are reminiscent of vertebrate gastrulation. These findings may facilitate the use of stem cells such as iPS cells for therapeutic purposes and as in vitro models for drug discovery and toxicity testing.

In particular, the present disclosure provides for the production of enriched cultures of, in one embodiment, iPS cell derived, definitive endoderm in the presence of a growth media. In contrast to current in vitro protocols for definitive endoderm differentiation in complex media with at least 3 days of differentiation, the present disclosure provides for an improved media for faster induction (e.g., over about 2 days) of mature definitive endoderm, which also gives rise in one embodiment to pancreatic progenitors. In addition, the medium composition allows for greater differentiation on many different stem cells, e.g., many different iPS cell lines, resulting in a platform for drug screening and basic research from different genetic backgrounds. The data also indicate that the temporal sequence of gene expression characteristic of iPS cell differentiation to definitive endoderm is similar to the transitions that occur in the course of definitive endoderm differentiation during vertebrate gastrulation, therefore resulting in a model to mimic and study human development.

Thus, the present disclosure provides compositions and methods to generate definitive endoderm from stem cells such as human iPS cells for, in one embodiment, the production of pancreas insulin producing cells. In one embodiment, the medium comprises a plurality of the components in STMX-DE.

In one embodiment, the disclosure provides a cell medium composition, e.g., a serum free composition, comprising a plurality of the following: one or more base tissue culture media; an amount of glutamine or an analog thereof, an amount of one or more lipids; an amount of one or more non-essential amino acids; an amount of one or more thiols; an amount of one or more of insulin or insulin like compounds, transferrin or selenium; or an amount of polyvinylalcohol, or any combination thereof. In one embodiment, the composition, e.g., a serum free composition includes three or more of: base tissue culture media; glutamine or an analog thereof; one or more lipids; one or more non-essential amino acids; one or more thiols; one or more of insulin or insulin like compounds, transferrin or selenium; or polyvinylalcohol. In one embodiment, the composition, e.g., a serum free composition includes a base tissue culture medium and glutamine or an analog thereof, and two or more of: a lipid; a non-essential amino acid; a thiol; insulin or insulin like compound; transferrin; selenium; or a polyvinylalcohol. In one embodiment, the composition, e.g., a serum free composition, includes a base tissue culture medium, glutamine or an analog thereof, and non-essential amino acids and optionally one or more of a lipid; a thiol; insulin or insulin like compounds, transferrin and/or selenium; or a polyvinylalcohol. In one embodiment, the composition, e.g., a serum free composition, includes a base tissue culture medium, glutamine or an analog thereof, and insulin or insulin like compounds, transferrin or selenium and optionally one or more of a lipid; a thiol; a non-essential amino acid, or a polyvinylalcohol. In one embodiment, the composition is serum-free.

In one embodiment, the composition further comprises an amount of Activin, e.g., Activin A or Activin A/B. In one embodiment, the amount of Activin A is about 50 ng/mL to about 150 ng/mL. In one embodiment, the amount of Activin A is about 75 ng/mL to about 125 ng/mL. In one embodiment, the amount of Activin A is about 80 ng/mL to about 125 ng/mL. In one embodiment, the composition further comprises an amount of an activator of WNT signaling or an inhibitor of glycogen synthase kinase 3. In one embodiment, the activator of WNT signaling or inhibitor of glycogen synthase kinase 3, comprises one or more of 3F8, A 1070722, AR-A 014418, BIO, BIO-acetoxime, CHIR 99021, CHIR 99021 trihydrochloride, Indirubin-3′-oxime, IM-12, MeBIO, bikini, kenpaullone, L803, lithium carbonate, NSC 693898, SB216763, SB415286, TC-G 24, TCS 2002, TCS 21311, TDZD-8, TWS 119, Tideglusib, AZD2858, AZD1080, or CHIR98014, or any combination thereof. In one embodiment, the inhibitor of glycogen synthase kinase 3 comprises CHIR99021, LY2090314 or SB216763. In one embodiment, the inhibitor of glycogen synthase kinase 3 is an aminopyridine. In one embodiment, the amount of CHIR99021 is about 1 μM to about 3 μM. In one embodiment, the amount of CHIR99021 is about 1.5 μM to about 2.5 μM. In one embodiment, the amount of CHIR99021 is about 0.5 μM to about 5 μM. In one embodiment, the amount of the inhibitor of glycogen synthase kinase 3 is about 0.5 μM to about 2.5 μM. In one embodiment, the amount of the inhibitor of glycogen synthase kinase 3 is about 2 μM to about 4 μM. In one embodiment, the amount of the inhibitor of glycogen synthase kinase 3 is about 1.5 μM to about 4 μM. In one embodiment, the analog of glutamine comprises a dipeptide, e.g., L-alanyl-L-glutamine dipeptide. In one embodiment, the amount of glutamine or the analog thereof in the composition comprises about 0.1% vol/vol to about 5% vol/vol. In one embodiment, the amount of glutamine or the analog thereof in the composition comprises about 100 mM to 300 mM glutamine or the analog thereof. In one embodiment, the amount of glutamine or the analog thereof in the composition comprises about 0.5% vol/vol to about 3% vol/vol. In one embodiment, the amount of glutamine or the analog thereof in the composition comprises about of about 150 mM to 400 mM glutamine or the analog thereof. In one embodiment, the amount of glutamine or the analog thereof in the composition comprises about 50 mM to about 250 mM. In one embodiment, the amount of the one or more lipids comprises about 0.1% vol/vol to about 5% vol/vol. In one embodiment, the lipids are a lipid concentrate. In one embodiment, the amount of the one or more lipids comprise about 0.5% vol/vol to about 3% vol/vol. In one embodiment, the one or more thiols comprise monothioglycerol. In one embodiment, the amount of one or more thiols comprise about 100 μM to about 900 μM. In one embodiment, the amount of one or more thiols comprise about 300 μM to about 800 μM. In one embodiment, the amount of the thiol comprise about 300 μM to about 600 μM. In one embodiment, the composition comprises a combination of insulin, transferrin and selenium. In one embodiment, the amount of the combination of insulin, transferrin and selenium in the composition is about 0.5% to about 5% vol/vol, where the insulin is about 500 mg/L to about 1500 mg/L, e.g., 1000 mg/mL, the transferrin is about 400 mg/L to about 700 mg/L, e.g., about 550 mg/L, and the selenium is about 0.3 to about 1.0 mg/L, e.g., about 0.67 mg/L. In one embodiment, the polyvinylalcohol has a molecular weight of about 80,000 to about 105,000. In one embodiment, the polyvinylalcohol has a molecular weight of about 85,000 to about 100,000. In one embodiment, the amount of the polyvinyl alcohol comprises about 0.1 mg/mL to about 10 mg/mL. In one embodiment, the amount of the polyvinyl alcohol comprises about 0.5 mg/mL to about 5 mg/mL. In one embodiment, one of the base tissue culture media comprises DMEM, e.g., DMEM-F12. In one embodiment, one of the base tissue culture media comprises Iscove's modified Dulbecco medium (IMDM). In one embodiment, the composition further comprises stem cells, e.g., induced pluripotent stem cells.

Further provided is a method to induce definitive endoderm differentiation in stem cells. The method includes combining stem cells, the composition described herein, and an amount of Activin and an amount of a GSK3 inhibitor effective to induce primitive streak formation in the cells, thereby providing a mixture comprising cells with primitive streak formation. The mixture or a portion thereof of the cells with primitive streak formation are combined with an amount of Activin and an amount of an inhibitor of AMP activated kinase effective to induce definitive endoderm differentiation, thereby providing definitive endoderm cells. In one embodiment, the stem cells are induced pluripotent stem cells. In one embodiment, the method further comprises treating the definitive endoderm cells with one or more agents and/or under conditions, e.g., temperature, compounds including growth factors or length of time, that provide for posterior foregut progenitor formation. In one embodiment, the method further comprises treating posterior foregut progenitor cells with one or more agents and/or under conditions that provide for pancreatic progenitors. In one embodiment, the method further comprises treating pancreatic progenitors with one or more agents and/or under conditions that provide for endocrine progenitors. In one embodiment, the method further comprises treating endocrine progenitors with one or more agents and/or under conditions that provide for insulin producing cells. In one embodiment, the induction of primitive streak formation occurs in about 1 day. In one embodiment, the induction of primitive streak formation occurs in less than about 2 days. In one embodiment, the induction of definitive endoderm formation occurs in about 1 day. In one embodiment, the induction of definitive endoderm formation occurs in less than about 2 days. In one embodiment, the induction of primitive streak formation and the induction of definitive endoderm formation occurs in about 2 days. In one embodiment, the induction of primitive streak formation and induction of definitive endoderm formation occurs in less than 3 days.

### BRIEF DESCRIPTION OF THE DRAWINGS

FIG. 1 shows a comparison of four exemplary cell lines undergoing definitive endoderm induction at day 1 in various growth media.

FIG. 2 shows a comparison of four exemplary cell lines undergoing definitive endoderm induction at day 2 in various growth media.

FIG. 3 shows a comparison of 1 cell line (iPSC-6) in day 2 of endoderm induction in various exemplary growth media.

FIG. 4 shows the presence of a biomarker SOX17 (immunostaining for hSOX17 protein) which is indicative of robust definitive endoderm formation.

FIG. 5 shows the gene expression pattern of cells during days 1 to 3 on STMX-DE media compared with GIBCO media at day 2 of differentiation.

### DETAILED DESCRIPTION

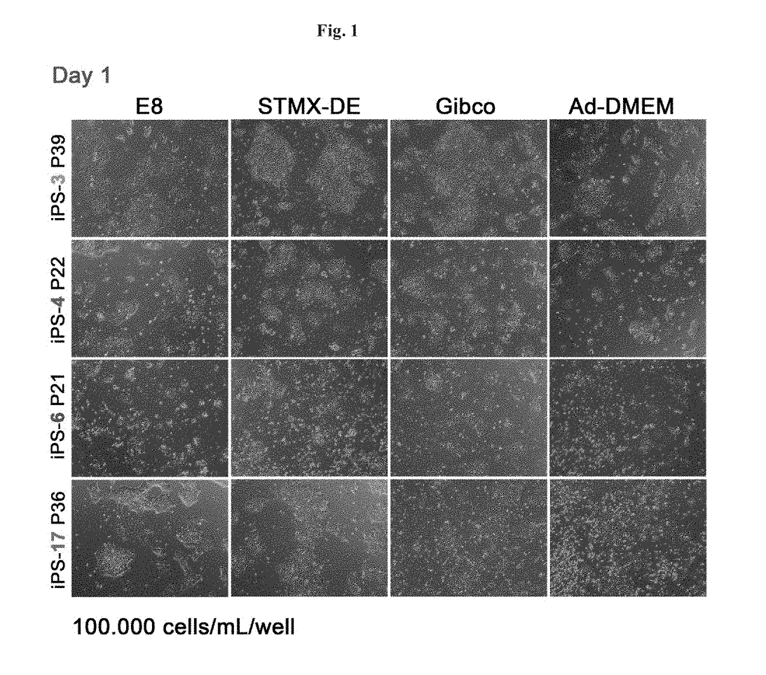

Referring to FIG. 1, this shows a comparison of four cell lines undergoing definitive endoderm induction at day 1 in various exemplary growth media (STMX-DE based media, GIBCO and Advanced-DMEM). Undifferentiated cells are shown in E8 media in vitronectin-coated plates. Colonies of pluripotent iPSCs are distributed evenly reaching no more than 40% confluency at day 1. The first day of differentiation concerns the primitive streak induction observed in vivo during development. All lines show growth on STMX-DE and GIBCO media, but undesirable morphology in Advanced-DMEM media.

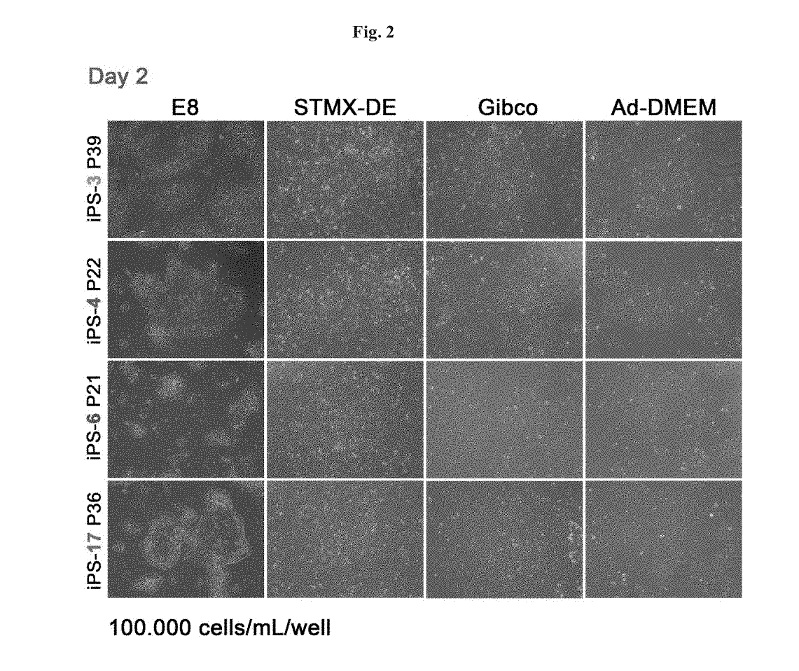

FIG. 2 shows a comparison of four exemplary cell lines undergoing definitive endoderm induction at day 2 in various growth media (STMX-DE, GIBCO and Advanced-DMEM). All cell lines perform better in STMX-DE media compared with GIBCO media. Ad-DMEM is not a good media for DE induction in iPS cells.

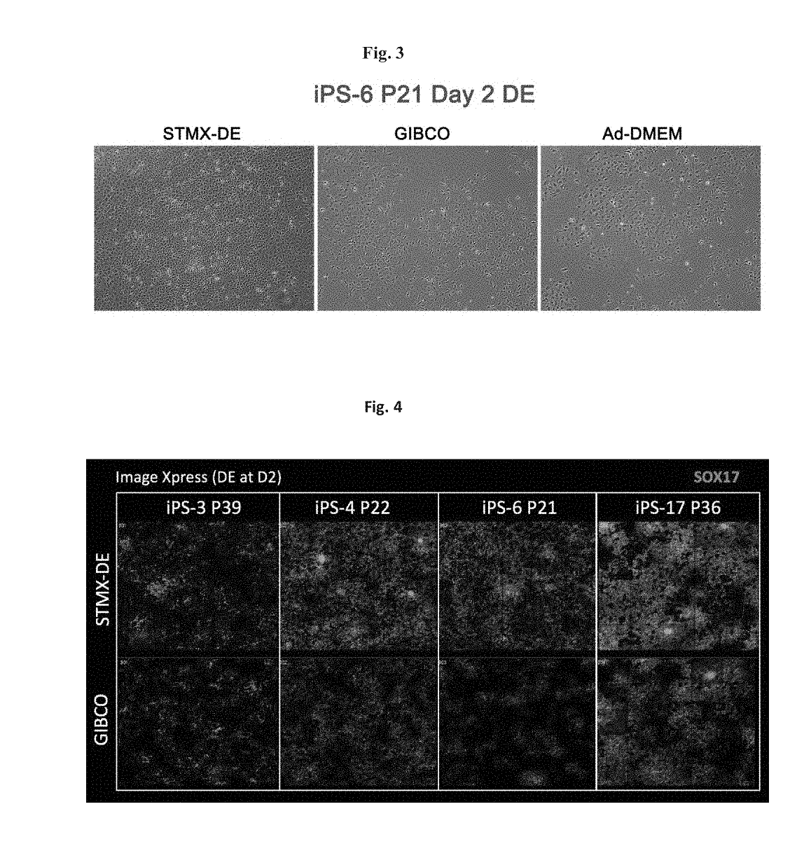

FIG. 3 shows a comparison of 1 cell line (iPSC-6) in day 2 of endoderm induction in various growth media. Enhanced differentiation and high yield of fully differentiated definitive endoderm cells was observed in STMX-DE media compared with GIBCO media. A typical morphological feature of definitive endoderm cells observed in STMX-DE is the appearance of highly compacted polygonal cells. In contrast, Ad-DMEM provides for non-efficient differentiation to epitheloid cells, which is not characteristic of definitive endoderm progenitors.

FIG. 4 shows the presence of a biomarker SOX17 (immunostaining for hSOX17 protein) indicative of robust definitive endoderm formation. Images are captured with Image-Xpress high-throughput imaging system: 25 images (10× objective) per iPSC line and condition. All lines show an even expression of SOX17, with different efficiencies depending on the iPSC line. Some lines that are refractory for differentiation (iPS-6) in GIBCO media show a higher number of SOX17 positive cells in STMX-DE media.

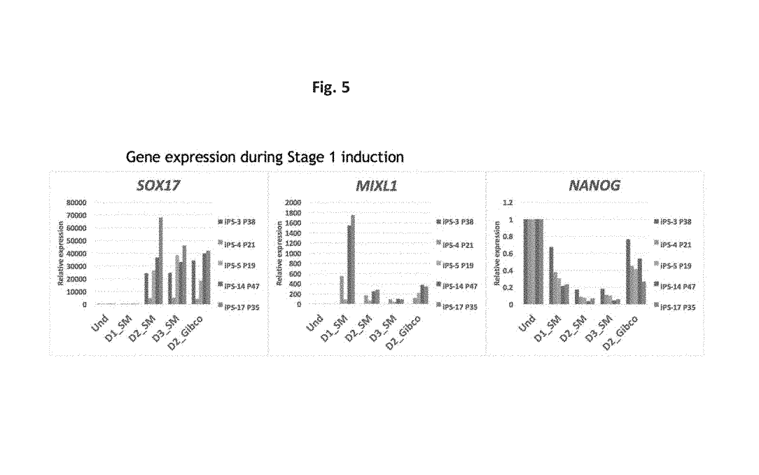

FIG. 5 shows the gene expression pattern of cells during days 1 to 3 on STMX-DE media compared with GIBCO media at day 2 of differentiation. Results are representative of qPCR results using undifferentiated controls in E8 media as reference control for each line. Sox17, the master gene for definitive endoderm differentiation, is highly expressed in all lines, with subtle differences in expression levels, depending on the line, as expected due to genetic differences. MixL1 is expressed at day 1 of differentiation in STMX-DE media, confirming mesendoderm induction in all cell lines. Absence of MixL1 expression at day 2 and 3 on STMX-DE media confirms the absence of mesoderm progenitors during definitive endoderm induction. Nanog expression is downregulated in STMX-DE media, confirming differentiation of cells; however, in GIBCO media, residual expression of Nanog would infer an impaired differentiation capacity.

### Exemplary Embodiments

In one embodiment, a cell medium composition comprises one or more base tissue culture media, an amount of glutamine or an analog thereof, e.g., about 0.1% vol/vol to about 5% vol/vol, and an amount of one or more non-essential amino acids, and optionally one or more of an amount of one or more lipids; an amount of one or more thiols; an amount of one or more of insulin, transferrin and/or selenium; or an amount of polyvinylalcohol, or any combination thereof. In one embodiment, the composition further comprises an amount of activin, e.g., about 50 ng/mL to about 150 ng/mL. In one embodiment, the composition further comprises an amount of an activator of WNT signaling or an inhibitor of glycogen synthase kinase 3. In one embodiment, the composition further comprises an amount of CHIR99021, e.g., about 1 μM to about 3 μM. In one embodiment, the composition comprises about 0.1% vol/vol to about 5% vol/vol lipids. In one embodiment, the composition comprises a thiol such as monothioglycerol, e.g., at about 100 μM to about 900 μM. In one embodiment, the composition comprises polyvinylalcohol with a molecular weight of about 80,000 to about 105,000, optionally at about 0.1 mg/mL to about 10 mg/mL. In one embodiment, the composition comprises DMEM. In one embodiment, the composition comprises DMEM-F12. In one embodiment, the composition comprises Iscove's modified Dulbecco medium (IMDM). In one embodiment, the composition further comprises stem cells, for instance, human stem cells including human induced pluripotent stem cells.

In one embodiment, a cell medium composition comprises one or more base tissue culture media, an amount of glutamine or an analog thereof, e.g., about 0.1% vol/vol to about 5% vol/vol, and an amount of selenium, and optionally one or more of an amount of one or more lipids; an amount of one or more thiols; an amount of one or more non-essential amino acids; an amount of transferrin; an amount of insulin; or an amount of polyvinylalcohol, or any combination thereof. In one embodiment, the composition further comprises an amount of Activin, e.g., about 50 ng/mL to about 150 ng/mL. In one embodiment, the composition further comprises an amount of an activator of WNT signaling or an inhibitor of glycogen synthase kinase 3. In one embodiment, the composition further comprises an amount of CHIR99021, e.g., about 1 μM to about 3 μM. In one embodiment, the composition comprises about 0.1% vol/vol to about 5% vol/vol lipids. In one embodiment, the composition comprises a thiol such as monothioglycerol, e.g., at about 100 μM to about 900 μM. In one embodiment, the composition comprises polyvinylalcohol with a molecular weight of about 80,000 to about 105,000, optionally at about 0.1 mg/mL to about 10 mg/mL. In one embodiment, the composition comprises DMEM. In one embodiment, the composition comprises DMEM-F12. In one embodiment, the composition comprises Iscove's modified Dulbecco medium (IMDM). In one embodiment, the composition ition further comprises stem cells, for instance, induced pluripotent stem cells.

In one embodiment, a method to induce definitive endoderm differentiation in stem cells is provided. The method include combining stem cells, one of the base media disclosed herein, and an amount of activin and an amount of a GSK3 inhibitor effective to induce primitive streak formation in the cells, thereby providing a mixture comprising cells with primitive streak formation. That mixture or a portion thereof or the cells with primitive streak formation, is combined with one of the media disclosed herein, and an amount of activin and an amount of an inhibitor of AMP activated kinase effective to induce definitive endoderm differentiation, thereby providing definitive endoderm cells. In one embodiment, the stem cells are induced pluripotent stem cells. In one embodiment, the definitive endoderm cells are treated with one or more agents or under conditions that provide for posterior foregut progenitor formation. In one embodiment, the posterior foregut progenitor cells are treated with one or more agents or conditions that provide for pancreatic progenitors. In one embodiment, the pancreatic progenitors are treated with one or more agents or conditions that provide for endocrine progenitors. In one embodiment, the endocrine progenitors are treated with one or more agents or conditions that provide for insulin producing cells. In one embodiment, the induction of primitive streak formation occurs in about 1 day. In one embodiment, the induction of primitive streak formation occurs in less than about 2 days. In one embodiment, the induction of definitive endoderm formation occurs in about 1 day. In one embodiment, the induction of definitive endoderm formation occurs in less than about 2 days. In one embodiment, the induction of primitive streak formation and the induction of definitive endoderm formation occurs in about 2 days. In one embodiment, the induction of primitive streak formation and induction of definitive endoderm formation occurs in less than 3 days. In one embodiment, the stem cells are combined with the medium that has the amount of activin and the amount of a GSK3 inhibitor effective to induce primitive streak formation in the cells. In one embodiment, the cells with primitive streak formation or the mixture and combined with the medium that has the amount of activin and the amount of an inhibitor of AMP activated kinase effective to induce definitive endoderm differentiation.

Table 1 shows a comparison of exemplary media (STMX-DE) versus media disclosed in Cho et al. (2012).

TABLE 1

| Definitive | | | | | |
|---|---|---|---|---|---|
| Endoderm Recipe | Disclosed | | | | |
| Cytokines & Small | Media | | Cho et all 2012 | | |
| molecules | Day 1 | Day 2 | Day 1 | Day 2 | Day 3 |
| Activin A | ✓ | ✓ | ✓ | ✓ | ✓ |
| BMP4 | X | X | ✓ | ✓ | ✓ |
| FGF2 | X | X | ✓ | ✓ | ✓ |
| LY294002 | X | X | ✓ | ✓ | ✓ |
| CHIR99021 | ✓ | X | X | X | X |
| Dorsomorphin | X | ✓ | X | X | X |
| DMEM/F12 | ✓ | ✓ | ✓ | ✓ | ✓ |
| IMDM | ✓ | ✓ | ✓ | ✓ | ✓ |
| ITS 100X | ✓ | ✓ | X | X | X |
| Glutamax | ✓ | ✓ | ✓ | ✓ | ✓ |
| Lipid 100X | ✓ | ✓ | ✓ | ✓ | ✓ |
| Monohioglycerol | ✓ | ✓ | ✓ | ✓ | ✓ |
| NEAA | ✓ | ✓ | X | X | X |
| Polyvinylalcohol | ✓ | ✓ | ✓ | ✓ | ✓ |
| Pen/Strep | X | X | ✓ | ✓ | ✓ |
| Insulin | X | X | ✓ | ✓ | ✓ |
| Transferrin | X | X | ✓ | ✓ | ✓ |
| Vitronectin 1 mL | ✓ | ✓ | X | X | X |
| Gelatine and 10% FBS | X | X | ✓ | ✓ | ✓ |

To obtain DE cells (Stage 1), Primitive Streak (PS) formation is induced at day 1 of differentiation by treating cells for 24 hours with 100 ng/mL Activin A (Stemgent, or TOCRIS), 2 μM CHIR99021, and a GSK-3 inhibitor (TOCRIS). After PS commitment, cells were treated for another 24 hours during day 2 of differentiation with 100 ng/mL Activin A, and 100 nM Dorsomorphin (Axon Medchem).

For Stage 1 differentiation, cells were grown in STMX-DE media (that contains DMEM-F12 (GIBCO) and IMDM (GIBCO) mixed 1:1, 1% vol/vol Glutamax, 1% vol/vol concentrated lipids (GIBCO), 450 μM Monothioglycerol (Sigma), 1% Insulin-Transferrin-Selenium ITS supplement (GIBCO), 1% vol/vol non-essential amino acids (NEAAs, GIBCO) and 1 mg/mL Polyvinylalcohol M_{w} 89,000-98,000, 99+% hydrolyzed (SIGMA)). PVA is dissolved overnight at 4° C.

Cells are grown in treated-plates which were coated for 30 minutes (room temperature) with Vitronectin (Invitrogen) diluted in DPBS without Ca and Mg (GIBCO) 1:100 (final concentration: 5 μg/mL).

Day 1: Feed the cells with: STMX-DE media+Activin A (100 ng/mL)+CHIR99021 (2 μM).

Day 2: Refeed the cells with: STMX-DE media+Activin A (100 ng/mL)+Dorsomorphin (100 nM).

### Exemplary Media:

### STMX-DE

DMEM-F12 (GIBCO)/IMDM (GIBCO) mixed 1:1
1% Glutamax (vol/vol)
1% non-essential amino acids (NEAAs, GIBCO) (vol/vol)
1% concentrated lipids (GIBCO) (vol/vol)
1% Insulin-Transferrin-Selenium: ITS supplement (GIBCO)
450 uM Monothioglycerol (Sigma)
1 mg/mL Polyvinylalcohol M_{w} 89,000-98,000, 99+% hydrolyzed (SIGMA)
   STMX-DE-1: STMX-DE+100 ng/mL Activin A (Stemgent, or TOCRIS), and 2 μM CHIR99021 GSK-3 inhibitor (TOCRIS).
   STMX-DE-2: STMX-DE+100 ng/mL Activin A (Stemgent, or TOCRIS), and 100 nM Dorsomorphin (Axon Medchem).

In summary, an approach to produce highly enriched cultures of definitive endoderm progenitors from iPS cells is provided. This work suggests that differentiation of iPS cells may serve as a model of early vertebrate development. The production of iPS cell-derived definitive endoderm is also a step in generating scientifically and therapeutically useful cells of the definitive endoderm lineage, such as hepatocytes and pancreatic endocrine and exocrine cells.

### REFERENCES

Cho et al. Development. 2012 Aug. 15; 139(16): 2866-2877.
Loh et al. Cell Stem Cell. 2014 Feb. 6; 14(2): 237-252.
Xu et al. Mech Dev. 2011 September-December; 128(7-10):412-27.
McGaugh et al. J. Vis. Exp. 2017 Mar. 7; (121
Ninomiya et al. In Vitro Cell Dev. Biol. Anim. 2015 January; 51(1):1-8
Yabe et al. J. Diabetes. 2017 February; 9(2): 168-179.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A cell medium composition, comprising:
a) one or more base tissue culture media;
an amount of glutamine or an analog thereof;
an amount of one or more lipids;
an amount of one or more non-essential amino acids;
an amount of one or more thiols;
an amount of one or more of insulin, transferrin or selenium; and
an amount of polyvinylalcohol; or
b) one or more base tissue culture media;
an amount of glutamine or an analog thereof;
an amount of one or more non-essential amino acids; and optionally an amount of an inhibitor of glycogen synthase kinase 3 or an amount of an inhibitor of AMP activated protein kinase; or
c) one or more base tissue culture media;
an amount of glutamine or an analog thereof;
an amount of selenium; and optionally an amount of an inhibitor of glycogen synthase kinase 3 or an amount of an inhibitor of AMP activated protein kinase.

2. The composition of claim 1 further comprising an amount of Activin, comprising an amount of an activator of WNT signaling or an inhibitor of glycogen synthase kinase 3.

3. The composition of claim 2 wherein the amount of Activin is about 50 ng/mL to about 150 ng/mL.

4. The composition of claim 1 wherein the activator of WNT signaling or the inhibitor of glycogen synthase kinase 3 comprises CHIR99021.

5. The composition of claim 4 wherein the amount of CHIR99021 is about 1 μM to about 3 μM.

6. The composition of claim 1 wherein the amount of glutamine or the analog thereof comprises about 0.1% vol/vol to about 5% vol/vol.

7. The composition of claim 1 wherein the amount of the one or more lipids comprises about 0.1% vol/vol to about 5% vol/vol.

8. The composition of claim 1 wherein the thiol comprises monothioglycerol.

9. The composition of claim 1 wherein the amount of the thiol comprises about 00 μM to about 900 μM.

10. The composition of claim 1 which comprises insulin, transferrin and selenium.

11. The composition of claim 1 wherein the polyvinylalcohol has a molecular weight of about 80,000 to about 105,000.

12. The composition of claim 11 wherein the amount of the polyvinyl alcohol comprises about 0.1 mg/mL to about 10 mg/mL.

13. The composition of claim 1 further comprising stem cells.

14. A method to induce definitive endoderm differentiation in stem cells, comprising:
combining stem cells, the medium of claim 1, and an amount of Activin and an amount of a GSK3 inhibitor effective to induce primitive streak formation in the cells, thereby providing a mixture comprising cells with primitive streak formation; and
combining the mixture or the portion thereof, or the cells with primitive streak formation, the medium of claim 1, and an amount of Activin and an amount of an inhibitor of AMP activated kinase effective to induce definitive endoderm differentiation, thereby providing definitive endoderm cells.

15. The method of claim 14 further comprising treating the definitive endoderm cells with one or more agents or under conditions that provide for posterior foregut progenitor formation.

16. The method of claim 15 further comprising treating posterior foregut progenitor cells with one or more agents or under conditions that provide for pancreatic progenitors.

17. The method of claim 16 further comprising treating pancreatic progenitors with one or more agents or under conditions that provide for endocrine progenitors.

18. The method of claim 17 further comprising treating endocrine progenitors with one or more agents or under conditions that provide for insulin producing cells.

19. The method of claim 14 wherein the stem cells are combined with the medium that has the amount of Activin and the amount of a GSK3 inhibitor effective to induce primitive streak formation in the cells.

20. The method of claim 14 wherein the mixture of cells with primitive streak formation is combined with the medium that has the amount of Activin and the amount of an inhibitor of AMP activated kinase effective to induce definitive endoderm differentiation.
